(19) United States
(12) Patent Application Publication
(10) Pub. No.: US 2008/0161709 A1
(43) Pub. Date: Jul. 3, 2008

(54) Breath Monitoring Apparatus and Method

(75) Inventor: Austen Peter Bradley, Lancashire (GB)

Correspondence Address: HUSCH BLACKWELL SANDERS LLP, 720 OLIVE STREET, SUITE 2400, ST. LOUIS, MO, 63101, US

(73) Assignee: NUTREN TECHNOLOGY LIMITED, Padiham (GB)

(21) Appl. No.: 10/598,610

(22) PCT Filed: May 19, 2005
(86) PCT No.: PCT/GB2005/001925

§ 371 Date: Oct. 13, 2006

(30) Foreign Application Priority Data
May 29, 2004 (GB) 0412096.0

(51) Int. Cl. A61B 5/08

(52) U.S. Cl. 600/532; 600/529; 600/539

(57) ABSTRACT

A breath monitoring apparatus is provided comprising a housing (4) on which is mounted, a means (6, 10, 12) to measure the volume of an inhaled breath of the user; and a means (8) to measure the gas content of an exhaled breath of a user. There is further provided a method of monitoring breaths, the method comprising the steps of calculating the volume of an inhaled breath of a user and calculating the gas content of an exhaled breath of the user. The apparatus and method relate in particular to indirect calorimetry.

### FIELD OF THE INVENTION

This invention relates to a breath monitoring apparatus and methods of monitoring breathing.

### BACKGROUND OF THE INVENTION

It is known to provide instruments which measure a breathing parameter of a user's breath, and which displays the result of the measurement for a user, sports scientist or doctor to review, for example.

In particular, devices are known for use in calorimetry, to study the energy of metabolism in humans and animals. Calorimetry is used, for example, for diagnosis of metabolic disorders and for calculating nutritional requirements of a subject. A useful calorimetric measure for nutritional and sports scientists, when assessing the health and fitness of a subject, is the volume of oxygen consumed at rest, and during or after physical exertion.

Indirect calorimetry often involves measuring the amount of carbon dioxide exhaled by a subject, which can in turn be used to calculate the oxygen consumption of a subject.

The measurement of the volume of oxygen consumed and/or the amount of carbon dioxide exhaled by a subjected are normally measured as a single reading, at rest, during or after physical exertion. Multiple measurements may be taken and plotted on a chart to indicate changes in oxygen and/or carbon dioxide measured over a period of time.

One problem with measuring parameters of breathing states, such as volume of oxygen consumed at rest and/or during or after physical exertion, is that the devices used to monitor the breathing parameter lead the user to “force” a breath, due to having to exhale and inhale through a breathing tube attached to the device. Thus the subject does not always breathe normally, and forces a breath from their lungs, or into their lungs as an instinctive reaction. The measurement of forced breaths does not give an accurate indication of the breathing parameter measured, as the breath will generally have a greater volume, speed, oxygen and/or carbon dioxide content.

It would therefore be advantageous to provide a breath monitoring device which prevents or mitigates calculation of a breathing parameter of a forced breath.

It would be furthermore advantageous to provide a breath monitoring device which allows forced breaths to be inputted, but which can calculate reliable results therefrom, allowing for the extra volume created by a forced breath.

In many breath monitoring devices, the device is arranged to take a single measurement and extrapolate it over an extended period. It is desirable that the measurement taken is as representative of the whole extended period as possible. One method to achieve this is by taking a measurement over several breaths, this can be averaged out to get a representative value. Another method would be to ensure that any breath taken is as normal as possible.

It is normal during utilisation of breath monitoring devices to measure the exhaled breath and make the assumption that the inhaled breath is made up of ambient air. There are essentially two measurements taken during this process. Firstly, the volume of exhaled gas is measured, and secondly, the content of exhaled gas is measured, preferably at the same time.

It is well known that when a user becomes conscious of their breathing, they control their breathing patterns and so this is not “normal” breathing. This is important when measuring breath, as when a user is asked to breathe into a breath monitoring device through a mask or mouthpiece, it immediately draws their attention to their breathing pattern and generally prevents the user from taking a normal exhaled breath.

There are essentially two basic phases of breathing, the inhalation phase and the exhalation phase. Measurement has generally been taken on the exhaled breath in known breath monitoring devices, but exhaled breath is easily distorted when a user thinks about breathing. In contrast, the inhalation phase is more of a reflex action and is more difficult to alter consciously.

It would therefore be advantageous to provide a breath monitoring device which utilises the more reflex action of the inhalation phase whilst enabling calculation of gas content of the exhalation phase in order to determine oxygen or carbon dioxide consumption.

It is therefore an aim of the preferred embodiments of the present invention to overcome or mitigate at least one problem of the prior art whether expressly disclosed herein or not.

### SUMMARY OF THE INVENTION

According to the first aspect of the present invention there is provided a breath monitoring apparatus comprising a housing on which is mounted:

a) a means to measure the volume of an inhaled breath of the user; and

b) a means to measure the gas content of an exhaled breath of a user.

According to a second aspect of the present invention there is provided a method of monitoring breaths, the method comprising the steps of:

a) calculating the volume of an inhaled breath of a user, and

b) calculating the gas content of an exhaled breath of the user.

Suitably, the breath monitoring apparatus is a calorimeter, and may be a direct calorimeter or an indirect calorimeter.

Preferably the device comprises a fluid inlet, which in use is arranged to allow passage of a user's breath into and out of the device.

Preferably the fluid inlet comprises a mouthpiece, integral with or connected to the fluid inlet. Suitably the mouthpiece is detachably connected to the fluid inlet, which enables the mouthpiece to be cleaned between uses.

The mouthpiece may comprise a mask arranged in use to be placed over at least the mouth of a user, and more preferably the mouth and nose of a user. Suitably the mask has securement means, arranged to secure the mask to a user's face. A mask is preferred as a mouthpiece as it encourages normal, unforced breathing from a user. Other mouthpieces, such as tubes, which are arranged to partially enter a user's mouth, may tend to encourage a user to force breaths into and/or out of a fluid inlet.

Preferably the means to measure the volume of an inhaled breath of a user comprises a flow sensor or flow meter.

Preferably the flow sensor comprises a moveable member, moveable by air pressure effected thereupon, and a movement sensor associated with the moveable member.

The moveable member may be a rotatable member, such as a rotor or paddle wheel, for example.

Alternatively, the moveable member may be a plunger, slidably mounted within the housing.

Suitably, the plunger is arranged to move slidably along the length of the housing.

Suitably, there is a substantially fluid-tight contact between the periphery of the moveable member and the interior surface of the housing.

The means to measure the volume of an inhaled breath of a user may for example comprise a mass flow meter, such as a Honeywell AWM 5104 VN Mass Flow Meter (supplied by Honeywell, USA), for example.

The means to measure the gas content of an exhaled breath of a user may comprise an oxygen sensor or a carbon dioxide sensor arranged to measure the oxygen content or carbon dioxide content of the exhaled breath, respectively. There may be more than one means to measure the gas content of an exhaled breath of a user, for example there may be both an oxygen sensor and a carbon dioxide sensor.

An example of a suitable oxygen sensor is an MOX-1(RTM) sensor supplied by City Technology Limited of Portsmouth, United Kingdom, or a City Technology 4OX-1 City Cell (RTM) sensor supplied by City Technology Limited of Portsmouth, United Kingdom.

Suitable carbon dioxide sensors include for example, a Gas Card II sensor supplied by Edinburgh Sensors, Edinburgh, United Kingdom.

The breath monitoring apparatus may further comprise a fluid outlet. Suitably, the fluid outlet is positioned such that excess exhaled breath passes out of the housing through the fluid outlet.

The apparatus may further comprise a collection chamber. The collection chamber may be in direct or indirect fluid flow communication with the fluid outlet. The collection chamber may comprise any suitable device, for example a bag or box. Suitably, the means to measure the volume of an inhaled breath of a user is located in the collection chamber. Suitably the collection chamber forms part of the housing of the apparatus and is therefore integral with the housing.

The apparatus may comprise an opening in the housing providing for external fluid to be inhaled through the apparatus. The opening may be sealed by a one-way valve, which valve allows fluid to be inhaled through the opening but prevents exhaled fluid from passing through the opening. Suitably the means to measure the gas content of an exhaled breath of a user is arranged to be non-operational during the use of the means to measure the volume of an inhaled breath of a user, and vice versa.

The apparatus in accordance with the invention may comprise means to calculate the respiratory oxygen consumption of a user. For example, the apparatus may comprise a computer or other electronic device for calculating the respiratory oxygen consumption utilising data provided by the means to measure the volume of an inhaled breath of a user and the means to measure the gas content of an exhaled breath of a user.

The method according to the invention may comprise measuring the volume of a plurality of inhaled breaths of a user and/or measuring the gas content of a plurality of exhaled breaths of a user. In some embodiments, the method may comprise measuring the volume of an inhaled breath of a plurality of inhaled breaths of a user, measuring the gas content of an exhaled breath or a plurality of exhaled breaths of a user, storing said measurements as a reference, and repeating steps (a) and (b) and comparing to the reference. The apparatus according to the invention may comprise means to connect the apparatus to a display screen or other visual indication device. Preferably the display screen is a television, LCD screen or computer monitor.

The measurement of the volume of inhaled breath and gas content of exhaled breath affords efficient monitoring of a user's breath, whilst allowing for forced exhalation. As volume of breath is only monitored during inhalation, which is generally a more natural reflex action, discrepancies due to monitoring of exhaled volume, if the exhaled breath is forced, are circumnavigated. The measurement of exhaled gas content is independent of the volume of the exhaled breath, as the concentrations of different gasses in the exhaled breath will be maintained, regardless of the volume of the breath.

As only one positive parameter is measured for each of the inhaled and exhaled breaths, data acquisition is simplified over and above prior art breath monitoring devices, as the apparatus in the present invention is collecting data from only one sensor at a time. This set up in turn helps to simplify the electronics of the device, resulting in both size and cost savings.

According to the third aspect of the present invention there is provided a method of monitoring breaths of the second aspect of the invention, using a breath monitoring apparatus of the first aspect of the invention.

### BRIEF DESCRIPTION OF THE DRAWINGS

The present invention will now be described, by way of example only, with reference to the following drawings, in which:

FIG. 1 is a schematic, partially cross-sectional, side view of part of a breath monitoring apparatus in accordance with the present invention, in a start position;

FIG. 2 is a schematic, partially cross-sectional, side view of the apparatus of FIG. 1 in an end position;

FIG. 3 is a schematic, partially cross-sectional, side view of a second embodiment of a breath monitoring apparatus in accordance with the present invention.

### DESCRIPTION OF THE PREFERRED EMBODIMENTS

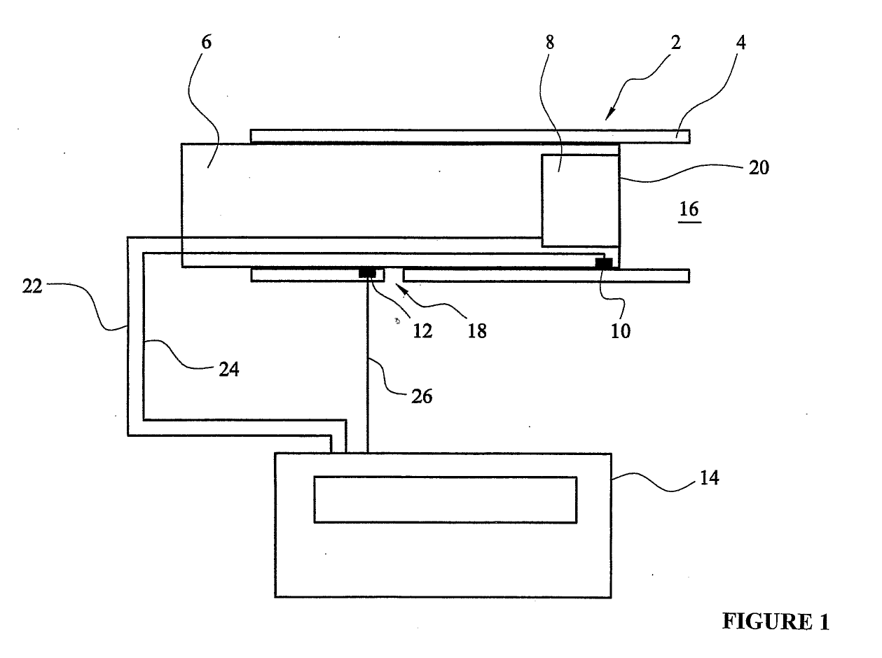

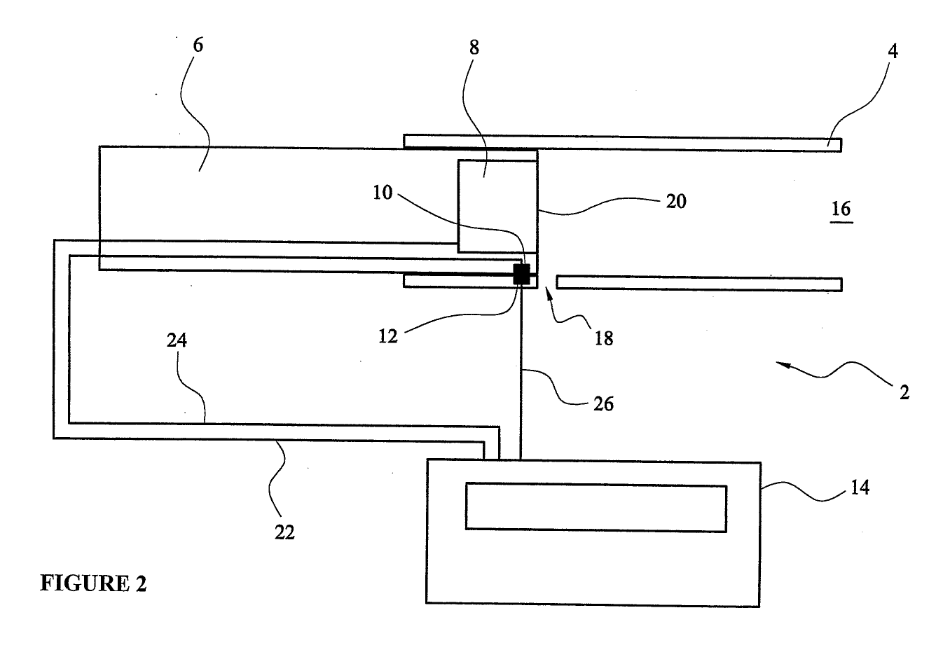

FIGS. 1 and 2 show part of a breath monitoring apparatus suitable in the form of a calorimeter, for calculating respiratory oxygen consumption comprising a housing 4, means to measure the volume of an inhaled breath in the form of a plunger 6 and a movement sensor provided by a first switch 10 and a second switch 12. The apparatus further comprises a means to measure the gas content of an exhaled breath in the form of an oxygen sensor 8. The apparatus 2 further comprises electronic calculation means 14.

The plunger 6 is slidably movable inside the housing 4, the interior of the housing 4 and the plunger 6 having the same cross-sectional shape. The relative dimensions of the periphery of the plunger 6 and the interior of the housing 4 being arranged such that there is a substantially fluid tight fit between the periphery of the plunger 6 and the interior of the housing 4.

The housing 4 comprises a fluid inlet 16 and a fluid outlet 18.

The oxygen sensor 8 is embedded in the end 20 of the plunger 6. The oxygen sensor is electrically connected to the electrical calculation means 14 by wire 22.

The first switch 10 of the movement sensor is embedded in a side of the plunger 4. The second switch 12 of the movement sensor is embedded in the interior wall of the housing 4. A wire 24 connects the first switch 10 with the electrical calculation means 14 and a wire 26 connects the second switch 12 with the electrical calculation means 14.

In use of the apparatus 2, the plunger 6 is arranged in the housing 4 at the start position as shown in FIG. 1. An exhaled breath is guided into the fluid inlet 16 of the housing 4. The end of the housing 4 providing the fluid inlet 16 may act as a mouthpiece for the apparatus 2. Alternatively, a separate mouthpiece (not shown) may be directly or indirectly connected to the housing 4.

The exhaled breath causes the plunger 6 to move away from the fluid inlet 16. The plunger 6 will continue to move in this direction until the end 20 of the plunger 6 passes the fluid outlet 18 to reach the end position as shown in FIG. 2. Thereafter, excess exhaled breath will pass out of the fluid outlet 18 and movement of the plunger 6 will cease.

Whilst the plunger 6 is moving from the start position to the end position, the oxygen sensor 8 is measuring the fraction of oxygen in the exhaled breath. The oxygen sensor 8 sends information of the measurement to the electrical calculation means 14 in the form of an electrical signal by means of wire 22.

The user may then inhale through the fluid inlet 16 in order to move the plunger back from the end position to the start position.

During movement of the plunger 6 from the end position to the start position, the switches 10, 12 of the movement sensor measure the time taken to move this set distance. This time is a measurement of the time taken to exhale a volume of inhaled breath. The switches 10, 12 send the measurement to the electrical calculation means 14 using wires 24 and 26.

The electrical calculation means 14 is programmed to calculate the oxygen consumption of the subject.

The method and calculation outlined above may be repeated one or more times, and the results may be integrated to provide an average value for the oxygen consumption.

The apparatus 2 is of a size and configuration such that it can be held and carried in the hand by an individual and connected to the electrical calculation means when desired.

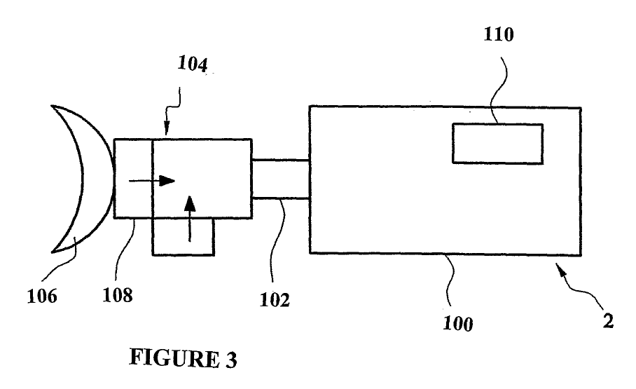

We refer now to FIG. 3 which illustrates a second preferred embodiment of the breath monitoring apparatus 2 of the invention. The breath monitoring apparatus 2 comprises a housing 100 in which is housed a means to measure the volume of an inhaled breath of a user, and means to measure the gas content of an exhaled breath of a user (not shown). The means to measure the gas content of the exhaled breath comprises a City Technology 4OX-1 City Cell Oxygen Meter, and the means to measure the volume of the inhaled breath comprises a Honeywell AWM 5104 VN Mass Flow Meter. The housing 100 also includes electronic calculation means to calculate the respiratory oxygen consumption based on measurements of inhaled volume and exhaled gas content.

Extending from one end of the housing 100 is a fluid inlet 102. A detachable mouthpiece 104 is connected to the fluid inlet 102. The mouthpiece 104 includes a t-valve 108, which allows exhaled breath to enter the fluid inlet 102, and into the device 2, but allows air from outside the device 2 to be inhaled through the t-valve when a user inhales. The mouthpiece 104 includes the mass flow meter, for measuring the volume of inhaled breath of a user, as inhaled breath is drawn through the t-valve 108. The distal end of the t-valve includes a flexible mask 106, arranged to be connected over the mouth of a user.

Usage of the device 2 shown in FIG. 3 is similar to that shown in FIGS. 1 and 2. The user first connects the detachable mouthpiece 104 to the fluid inlet 102 of the housing 100 of the device 2. When it is desired to monitor the respiratory oxygen consumption of a user, the user places the facemask 106 over his or her mouth, and begins to breathe into the mouthpiece 104. As a user inhales, air from outside of the device enters the t-valve 108, and triggers activation of the mass flow meter (not shown), which transmits data to the electronic calculation means, in order to calculate volume of inhaled air. As a user exhales, air from the user passes through the t-valve 108, through the fluid inlet 102 and is passed over the oxygen sensor (not shown) within the housing 100. Data from the oxygen sensor is passed to the electronic calculation means. The electronic calculation means then converts the inhaled volume and exhaled gas content data into a reading of respiratory oxygen consumption. The reading may be displayed on a display screen 110 on the device.

In alternative embodiments of the breath monitoring device of the invention, there may be a means for the device to prevent a user from operating the device if an error is detected. The device may also comprise means for a user to manually override the operation prevention means, such as a manual switch operable by user, for example. The device may comprise means for a user to bypass or ignore one or more breath measurements, for example if the user knows that they have purposely forced a breath, or believe a breath to be not representative of their normal breathing.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extend to any novel one, or any novel combination, of the features disclosed in this specification (including any accompanying claims, abstract and drawings), or to any novel one, or any novel combination, of the steps of any method or process so disclosed.

1. A breath monitoring apparatus comprising a housing on which is mounted:
a) a means to measure the volume of an inhaled breath of the user; and
b) a means to measure the gas content of an exhaled breath of a user.

2. A breath monitoring apparatus as claimed in claim 1 wherein the breath monitoring apparatus is a calorimeter.

3. A breath monitoring apparatus as claimed in claim 1 wherein the means to measure the volume of an inhaled breath of a user comprises a flow sensor or flow meter.

4. A breath monitoring apparatus as claimed in claim 3 wherein the flow sensor comprises a moveable member, moveable by air pressure effected thereupon, and a movement sensor associated with the moveable member.

5. A breath monitoring apparatus as claimed in claim 4 wherein the moveable member is a plunger, slideably mounted within the housing of the apparatus.

6. A breath monitoring apparatus as claimed in claim 4 wherein there is a substantially fluid-tight contact between the periphery of the moveable member and the interior surface of the housing.

7. A breath monitoring apparatus as claimed in claim 1 wherein the means to measure the volume of an inhaled breath of a user comprises a mass flow meter.

8. A breath monitoring apparatus as claimed in claim 1 wherein the means to measure the gas content of an exhaled breath of a user comprises an oxygen sensor or a carbon dioxide sensor, arranged to measure the oxygen content or carbon dioxide content of the exhaled breath respectively.

9. A breath monitoring apparatus as claimed in claim 1 comprising an opening in the housing providing for external fluid to be inhaled through the apparatus.

10. A breath monitoring apparatus as claimed in claim 1 further comprising means to calculate the respiratory oxygen consumption of a user.

11. A method of monitoring breaths, the method comprising the steps of:
a) calculating the volume of an inhaled breath of a user, and
b) calculating the gas content of an exhaled breath of the user.

12. A method as claimed in claim 11 comprising measuring the volume of a plurality of inhaled breaths of a user and/or measuring the gas content of a plurality of exhaled breaths of a user.

13. A method as claimed in claim 11 comprising the steps of:
a) measuring the volume of an inhaled breath or a plurality of inhaled breaths of a user;
b) measuring the gas content of an exhaled breath or a plurality of exhaled breaths of a user;
c) storing said measurements as a reference, and repeating steps (a) and (b) and comparing them to the reference.

14. A method of monitoring breaths as claimed in claim 11 using a breath monitoring apparatus as claimed in claim 1.

15. A method of monitoring breaths as claimed in claim 11 using a breath monitoring apparatus as claimed in claim 6.

16. A method of monitoring breaths as claimed in claim 11 using a breath monitoring apparatus as claimed in claim 8.

17. A method of monitoring breaths as claimed in claim 11 using a breath monitoring apparatus as claimed in claim 9.

18. A method of monitoring breaths as claimed in claim 11 using a breath monitoring apparatus as claimed in claim 10.
